(19) United States
(12) Patent Application Publication
(10) Pub. No.: US 2023/0329820 A1
(43) Pub. Date: Oct. 19, 2023

(54) A Medical Instrument Cover Dispensing Apparatus and a Cover Storage Arrangement and Cover for Use Therewith

(71) Applicants: Arjang Agahi, Country Antrim (GB); Orang Agahi, Belfast (GB)

(72) Inventors: Arjang Agahi, Country Antrim (GB); Orang Agahi, Belfast (GB)

(21) Appl. No.: 18/022,386

(22) PCT Filed: Jun. 30, 2021
(86) PCT No.: PCT/EP2021/068074

§ 371 Date: Feb. 21, 2023

(30) Foreign Application Priority Data
Aug. 25, 2020 (GB) 2013286.6

(51) Int. Cl. A61B 50/30; A61B 46/10; A61B 7/02

(52) U.S. Cl. CPC A61B 50/30; A61B 46/10; A61B 7/02; A61B 2050/3013

(57) ABSTRACT

A medical instrument cover dispensing apparatus 10 comprising: a cover storage arrangement 11 configured to house one or more medical instrument covers 12, said one or more medical instrument covers 12 being in a first configuration when housed in the cover storage arrangement 11. A cover engagement and placement assembly 13 is provided and is configured to receive one of the one or more medical instrument covers 12, or portion thereof, and move said received medical instrument cover 12 such that said medical instrument cover 12 is placed in a second configuration. Wherein, in the second configuration, said medical instrument cover 12 is operably engagable by a medical instrument 76 to attach the medical instrument cover 12 to the medical instrument 76.

### FIELD OF THE INVENTION

This invention relates to a medical instrument cover dispensing apparatus and a cover storage arrangement and cover for use therewith.

### Background of the Invention

Medical personnel often utilise instruments which are not single use such that they are used on multiple patients. One example of such an instrument is a stethoscope. Use of a single instrument on multiple patients raises obvious issues in regards to contamination and safety. More specifically, bacteria, infectious matter, or other biological matter may be transferred from patient to patient. It is generally accepted practice to clean the instrument between uses; however this cleaning is often unregulated, and highly dependent on the motivation of the medical personnel. In many cases, this cleaning is poorly carried out, or not carried out at all. A known solution is to apply a cover to instruments such as stethoscopes; these covers typically comprise an elasticated membrane with a rolled rim. The rim is stretched over the patient contacting portion of the instrument before use, and discarded after use. However, these covers can be cumbersome to apply/fit, and still must be touched to complete application thus presenting risk of contamination. Some solutions may assist with the fitting of the covers; however such solutions are cumbersome to load/replenish.

It is desirable to provide a medical instrument cover dispensing apparatus which quickly and effectively applies covers to medical instruments and is easily loaded with said covers, it would also be desirable to provide a cover storage arrangement and cover for use therewith.

### Summary of the Invention

According to the invention there is provided a medical instrument cover dispensing apparatus comprising: a cover storage arrangement configured to house one or more medical instrument covers, said one or more medical instrument covers being in a first configuration when housed in the cover storage arrangement; and a cover engagement and placement assembly configured to receive one of the one or more medical instrument covers, or portion thereof, and move said received medical instrument cover such that said medical instrument cover is placed in a second configuration; wherein, in the second configuration, said medical instrument cover is operably engagable by a medical instrument to attach the medical instrument cover to the medical instrument.

Ideally, the medical instrument cover dispensing apparatus is configured to supply one medical instrument cover at a time such that a user can operably engage the medical instrument cover with a medical instrument in a manner which acts to cover at least a portion of said medical instrument without the user touching the medical instrument cover.

Advantageously, covers may be applied to the medical instrument without contact which could contaminate the cover.

Ideally, the cover storage arrangement is configured to store multiple medical instrument covers.

Preferably, the cover storage arrangement is a removable cover storage arrangement.

Preferably, the cover storage arrangement is a removable cover storage cartridge.

Advantageously, the medical instrument cover dispensing apparatus may be replenished with medical instrument covers by insertion of a single cartridge containing a plurality of medical instrument covers, thus preventing the need to reload loose medical instrument covers or uncontained stacks of medical instrument covers.

Ideally, the cover engagement and placement assembly configured to receive a medical instrument cover, or portion thereof, from the cover storage arrangement.

Preferably, the medical instrument cover dispensing apparatus further comprising a feed arrangement.

Ideally, the feed arrangement feeds medical instrument covers from the cover storage arrangement to the cover engagement and placement assembly.

Preferably, the medical instrument cover dispensing apparatus further comprises a housing element having an outer wall portion defining an interior housing space.

Ideally, the housing element comprises upper opening.

Preferably, the upper opening of the housing element is an opening which, when the apparatus is assembled for use, is proximal the end of the apparatus from which medical instrument covers are dispensed.

Ideally, the housing comprises a lower opening opposing the upper opening thereof.

Ideally, the outer wall portion of the housing element comprises a seat feature.

Preferably, the seat feature is configured to receive at least a portion of a medical instrument cover.

Ideally, in use, the cover storage arrangement and feed arrangement are disposed at least partially within the interior housing space of the housing element.

Preferably, the cover storage arrangement and feed arrangement are movably mountable within the interior housing space of the housing element.

Ideally, the cover engagement and placement assembly is movably mountable on the outer wall portion of the housing element.

Preferably, the medical instrument cover is a generally circular cover having a circular outer rib defining a central aperture.

Ideally, a membrane is attached to or formed integrally with said outer rib and spans the central aperture.

Preferably, the housing element, feed arrangement, and cover engagement and placement assembly are generally cylindrical elements configured to be operable with the generally circular medical instrument cover.

Ideally, the generally cylindrical housing element, feed arrangement, cover engagement and placement assembly, are disposed in a co-axial arrangement relative to each other.

Preferably, the cover storage arrangement is generally cylindrical and is disposed in a co-axial arrangement with the housing element, feed arrangement, cover engagement and placement assembly.

Ideally, the cover engagement and placement assembly and the feed arrangement are operably engageable with each other.

Preferably, the cover engagement and placement assembly and the feed arrangement are operably engageable with each other through a plurality of openings in the outer wall portion of the housing element.

Preferably, the plurality of openings in the outer wall of the housing element are slotted openings.

Ideally, movement of one of the cover engagement and placement assembly or feed arrangement causes movement of the respective other arrangement.

Preferably, the cover engagement and placement assembly and feed arrangement move as one relative to the housing element.

Ideally, the cover engagement and placement assembly comprises a cover receiving portion which extends at least partially across the upper opening of the housing element in a first configuration.

Preferably, the cover receiving portion is configured to receive an outer rib of a medical instrument cover.

Ideally, the cover engagement and placement assembly further comprises a base portion.

Preferably, the base portion of the cover engagement and placement assembly is in engagement with the feed arrangement through the openings in the housing element.

Preferably, the base portion of the cover engagement and placement assembly is in engagement with the feed arrangement via fastening means locatable through the slotted openings in the housing element.

Ideally, the slotted openings are shaped and dimensioned to guide/restrict movement of the fastening means and thus the feed arrangement and cover engagement and placement assembly which are both connected thereto.

Preferably, the cover receiving portion of the engagement and placement arrangement is in rotatable connection with the base portion thereof.

Ideally, a guide formation is provided on the outer surface of the outer wall portion of the housing element.

Preferably, the guide formation is shaped and dimensioned to guide movement of the cover receiving portion from a first configuration wherein at least a portion thereof extends at least partially across the upper opening of the housing element to a second configuration wherein the cover receiving portion is rotated outwardly of the housing element.

Ideally, said outward rotation occurs as the engagement and placement arrangement is moved downwards relative to the housing element.

Preferably, downwards movement refers to movement in the axial direction of the housing element in a direction towards the lower opening thereof.

Ideally, the guide formation comprises an arcuate guide formation.

Preferably, the arcuate guide formation projects from the outer surface of the housing element forming an arcuate surface which is engagable by the cover receiving portion and along which the cover receiving portion is guided.

Ideally, the cover receiving portion comprises urging means for urging the cover receiving portion inwards towards the outer surface of the outer wall portion of the housing element.

Preferably, the urging means is a biasing element.

Ideally, the urging means is a spring or wire element.

Preferably, the cover engagement and placement assembly comprises apertures therein and the spring or wire element of the cover receiving portion is receivable by said apertures to at least partially span the circumference of the cover engagement and placement assembly.

Alternatively, or in combination, the urging means of the cover engagement and placement assembly is provided by the elastically deformable nature of the material connecting the base portion of the cover engagement and placement assembly to the cover receiving portion thereof.

Ideally, the material and/or geometry of the rotatable connection between the base portion and cover receiving portion of the cover engagement and placement assembly is such that the cover receiving portion is biased by said connection towards the outer wall of the housing element.

Preferably, the rotatable connection, if deformed by application of a force, will revert to its undeformed state upon removal of said force.

Ideally, the undeformed state of the rotatable connection is a state wherein the attached cover receiving portion is in its first configuration.

Preferably, the outer rib of the medical instrument cover is an elastically deformable outer rib.

Ideally, the receiving portion of the cover engagement and placement assembly comprises a receiving cup sized and dimensioned to receive an outer rib of a medical instrument cover.

Ideally, the cover engagement and placement assembly is movable such that the cover receiving portion thereof is configured to pull the outer rib of an uppermost medical instrument cover from a position generally above the interior housing space of the housing element.

Preferably, the cover engagement and placement assembly is configured to deposit said outer rib into the seat feature formed on the outer wall portion of the housing element.

Ideally, the seat feature in the outer wall portion of the housing element is sized and dimensioned to receive an outer rib of a medical instrument cover.

Preferably, the membrane of the medical instrument cover covers the upper opening in the housing element when the outer rib of said medical instrument cover is locatable in the seat feature.

Ideally, the level of elasticity of the outer rib of the medical instrument cover is toleranced such that said outer rib becomes dislodged from the receiving cup of the receiving portion of the cover engagement and placement assembly and deposited into the seat feature.

Preferably, dislodging of the outer rib from the receiving cup occurs as the receiving cup moves to a position adjacent the seat feature.

Alternatively, or in conjunction, a rib removal feature is provided on the cover engagement and placement assembly which is adapted to disturb the outer rib such that it is actively dislodged from the receiving cup and deposited into the seat feature.

Ideally, the cover receiving portion comprises a plurality of rotatable hook elements disposed radially on the base portion.

Preferably, each rotatable hook element has a corresponding guide feature locatable on the outer wall of the housing element.

Ideally, each rotatable hook element comprises a corresponding rotatable connection.

Ideally, the plurality of rotatable hook elements are configured to move in unison between the first and second configurations of the cover receiving portion.

Preferably, the plurality of rotatable hook elements form a jaw like arrangement wherein the jaw like arrangement is closed in the first configuration and open in the second configuration of the cover receiving portion.

Ideally, the feed arrangement comprises an outer wall portion defining an inner through channel sized and dimensioned for receiving the cover storage element.

Preferably, the outer wall portion of the feed arrangement further comprises one or more cover retaining and release features at an upper end thereof.

Ideally, the cover retaining and release features are configured to selectably restrict upwards movement of the one or more medical instrument covers in the cover storage arrangement.

Ideally, the cover retaining and release features are configured to selectably restrict upwards movement of the one or more medical instrument covers in the cover storage arrangement when the feed arrangement is in a first configuration.

Preferably, when the feed arrangement is in a second configuration, the one or more cover retaining and release features release an uppermost medical instrument cover of the one or more medical instrument covers in the cover storage arrangement.

Ideally, release of the uppermost medical instrument cover of the one or more medical instrument covers in the cover storage arrangement by the at least one cover retaining and release feature is such that said uppermost medical instrument cover is permitted to travel towards and into contact with the cover engagement and placement assembly.

Preferably, the at least one cover retaining and release feature is rotatable between the first and second configurations of the feed arrangement.

Ideally, movement of the feed arrangement downwards relative to the housing element causes the outer wall of the housing element to engage the at least one cover retaining and release feature and cause rotation thereof inwardly towards the inner housing space of the housing element and into the first configuration from the second configuration.

Preferably, movement of the feed arrangement upwards relative to the housing element causes movement of the at least one cover retaining and release feature to at least partially move out of the upper opening of the housing element and above the outer wall of the housing element such that the at least one cover retaining and release feature may rotate outwardly relative to the inner housing space of the housing element and into the second configuration.

Ideally, the at least one cover retaining and release feature comprises urging means for urging the cover retaining and release feature outwards relative to the inner housing space of the housing element and into the second configuration.

Preferably, the urging means is a biasing element.

Ideally, the feed arrangement comprises a plurality of cover retaining and release features disposed radially on an upper end thereof.

Preferably, the plurality cover retaining and release features are configured to move in unison between the first and second configurations of the feed arrangement.

Preferably, the plurality of cover retaining and release features are disposed in a spaced apart radial arrangement on the upper edge of the feed arrangement and spacing members are locatable therebetween.

Preferably, the biasing element of the cover retaining and release features comprises a spring or wire element.

Ideally, the spacing members comprise throughbores therein through which the spring or wire element of the biasing element of the cover retaining and release features is threaded.

Preferably, the spring or wire element of the biasing element of the cover retaining and release features acts to exert a biasing force on the cover retaining and release features outwards relative to the inner housing space of the housing element.

Ideally, the cover retaining and release features are attachable to or formed integrally with the remainder of the feed arrangement about a connection which is elastically deformable and which, if deformed by application of a force, will revert to its undeformed state upon removal of said force. Preferably, the undeformed state of the cover retaining and release features is a state wherein the cover retaining and release features are in the first configuration of the feed arrangement.

Preferably, the housing element comprises a cover de-seat feature configured to urge the portion of a cover locatable in the seat feature of the housing element from said seat feature and into engagement with a medical instrument.

Preferably the cover de-seat feature comprises a plurality of rotatable instrument engagement features disposed radially around the outer wall of the housing element and projecting at least partially into the inner space of the housing element in a first configuration.

Ideally, the cover de-seat feature is rotatable into a second configuration wherein a portion of the cover de-seat feature which is aligned with the seat feature is rotated outwardly and into the seat feature such that a cover seated in the seat feature is urged therefrom.

Preferably, movement of the cover de-seat feature from the first configuration to the second configuration is effected by the insertion of a medical instrument into the upper opening of the housing element.

Ideally, a medical instrument inserted into the upper opening of the housing element contacts the portion of the cover de-seat feature which projects at least partially into the inner space of the housing element in the first configuration, and preferably further insertion forces the cover de-seat feature to rotate outwardly relative to the inner space of the housing element and at least partially into the seat feature such that a cover seated in the seat feature is urged therefrom.

Preferably, the cover de-seat feature comprises urging means for urging the cover de-seat feature inwardly relative to the inner housing space of the housing element when the cover de-seat feature is in the second configuration.

Ideally, the urging means of the de-seat feature is a biasing means.

Preferably, the biasing means of the de-seat feature is a de-seat spring or wire element.

Alternatively, the cover de-seat feature comprises a slidable de-seat member, the slidable de-seat member being slidable relative to the housing element and sized and dimensioned to urge a cover seated in the seat feature therefrom.

Ideally, the slidable de-seat member is slidably mountable relative to the housing element.

Preferably, the slidable de-seat member is slidably insertable at least partially into the upper opening of the housing element and slidable therewithin.

Ideally, the slidable de-seat member comprises a lip portion.

Ideally, the lip portion is selectably engagable with an upper end of the outer wall portion of the housing element.

Preferably, the lip portion of the slidable de-seat member is movable between first and second positions thereof as the slidable de-seat member sildes within the housing element.

Ideally, in the first position, the lip portion is locatable such that the seat feature is defined between the lip portion and an upper end of the outer wall portion of the housing element.

Preferably, in the second position, the lip portion is locatable such that it is in engagement with the upper end of the outer wall portion of the housing element.

Alternatively, in the second position, the lip portion is locatable such that the space between the lip portion and the upper end of the outer wall portion of the housing element is reduced when compared to the space therebetween when the lip portion is in the first position.

Preferably, when the lip portion is in a first position an outer rib of a medical instrument cover is receivable in the seat feature defined between the lip portion and an upper end of the outer wall portion of the housing element, and movement of the slidable de-seat member such that the lip portion thereof is in the second position causes the lip portion, and/or the upper end of the outer wall portion of the housing element, to urge the outer rib from the seat feature.

Ideally, the guide formations comprise apertures therein and the de-seat spring or wire element is threadable therethrough such that the spring or wire element at least partially encircles the outer surface of the outer wall of the housing element at the location thereof having the de-seat features such that said de-seat wire or spring element acts to exert a biasing force on the de-seat features inwardly relative to the inner housing space of the housing element.

Ideally, the plurality of rotatable instrument engagement features of the cover de-seat feature are attachable to or formed integrally with the remainder of the housing element about a connection which is elastically deformable and which, if deformed by application of a force, will revert to its undeformed state upon removal of said force.

Preferably, the undeformed state of the plurality of rotatable instrument engagement features of the cover de-seat feature is a state wherein the plurality of rotatable instrument engagement features are in the first configuration of the cover de-seat feature.

Ideally, removal of a medical instrument from the inner space of the housing element results in the cover de-seat feature returning to the first configuration.

Preferably, the cover storage apparatus is a cover storage cartridge, the cover storage cartridge comprising a cylindrical outer wall defining a cylindrical inner cover receiving space.

Ideally, the cover storage cartridge is sized and dimensioned for insertion into the feed arrangement.

Preferably, the cover storage cartridge is insertable through a lower opening in the feed arrangement.

Ideally, the feed arrangement comprises a stop feature which engages with the cover storage cartridge to stop the cover storage cartridge at a desired location as it is inserted into the feed arrangement.

Preferably, the stop feature is provided by a reduced interior diameter, or feature which reduces the interior diameter, proximal a top end of the feed arrangement.

Ideally, a cover urging element is provided, the cover urging element being sized and dimensioned to be insertable into a lower opening in the cover storage cartridge and into contact with medical instrument covers therein.

Preferably, the cover urging element is in operable engagement with a biasing feature to bias the medical instrument covers towards an upper opening of the cover storage cartridge.

Ideally, the biasing feature is a spring locatable between the cover urging element and a base of the housing element.

Preferably, the base of the housing element comprises a removable base which is attachable to the housing element to cover the lower opening thereof.

Ideally, the removable base is attachable to the housing element via a mutually engagable threaded connection.

Preferably, the biasing feature of the cover urging element is a spring which is compressed between the cover urging element and the base of the housing element.

Ideally, the cover storage cartridge comprises a sacrificial retaining element sized and dimensioned to cover the lower opening of the cover storage cartridge.

Preferably, the sacrificial retaining element prevents the medical instrument covers from falling from the lower opening of the cover storage cartridge cover before the cover storage cartridge has been installed in the medical instrument cover dispensing apparatus.

Ideally, the sacrificial retaining element is formed from a material which my be punctured, perforated, tom, or the like by the cover urging element as the cover urging element passes into the lower opening of the cover storage cartridge.

Ideally, the medical instrument cover dispensing apparatus further comprises a user engagable casing element.

Preferably, the user engagable casing element is in operable engagement with the cover engagement and positioning arrangement such that a user may move the cover engagement and positioning arrangement by moving the user engagable casing element.

Ideally, the user engagable casing element comprises a lower opening sized and dimensioned to receive the cover engagement and positioning arrangement and an upper opening to provide access for a medical instrument.

Preferably, the user engagable casing element is attachable to the base portion of the cover engagement and positioning arrangement via a mutually engagable threaded connection.

According to a second aspect of the invention there is provided a cover storage arrangement for use in a medical instrument cover dispensing apparatus, wherein the cover storage apparatus is a cover storage cartridge, the cover storage cartridge comprising a cylindrical outer wall defining a cylindrical inner cover receiving space sized and dimensioned to receive a plurality of medical instrument covers, the cover storage cartridge being sized and dimensioned for insertion into a feed arrangement of the medical instrument cover dispensing apparatus.

Ideally, a lower opening of the cover storage cartridge is sized and dimensioned to receive a cover urging element such that the cover urging element may contact medical instrument covers therein.

Preferably, the cover urging element is in operable engagement with a biasing feature to bias the medical instrument covers towards an upper opening of the cover storage cartridge.

According to a third aspect of the invention there is provided a medical instrument cover for use in a medical instrument cover dispensing apparatus, wherein the medical instrument cover comprises an outer rib defining a central aperture, and a membrane attached to or formed integrally of said outer rib and spanning the central aperture.

Ideally, the medical instrument cover is a generally circular cover.

Alternatively, the medical instrument cover is a generally oval cover.

Ideally, the medical instrument cover comprises a recess formed on the underside of the outer rib thereof.

Preferably, the recess in the outer rib is shaped to receive the upper portion of an outer rib or another medical instrument cover therein.

Advantageously, the medical instrument cover may more easily be stacked with other medical instrument covers in a stacked arrangement.

### BRIEF DESCRIPTION OF THE DRAWINGS

An embodiment of the invention is now described by way of example and with reference to the accompanying drawings in which:

FIG. 1 is a sectional view of a medical instrument cover dispensing apparatus;

FIG. 2 is a front view of the medical instrument cover dispensing apparatus of FIG. 1;

FIG. 3 is a sectional view of a housing element and cover engagement and placement assembly of a medical instrument cover dispensing apparatus wherein a cover receiving portion of the cover engagement and placement assembly is in a first configuration;

FIG. 4 is a perspective cut-away view of FIG. 3;

FIG. 5 is a sectional view of the cover engagement and placement assembly of FIG. 3;

FIG. 6 is a perspective cut-away view of FIG. 5;

FIG. 7 is a sectional view of a housing element and cover engagement and placement assembly of a medical instrument cover dispensing apparatus wherein a cover receiving portion of the cover engagement and placement assembly is between a first configuration and a second configuration;

FIG. 8 is a sectional view of the cover engagement and placement assembly of FIG. 7;

FIG. 9 is a perspective cut-away view of FIG. 7;

FIG. 10 is a perspective cut-away view of FIG. 8;

FIG. 11 is a further perspective cut-away view of FIG. 7;

FIG. 12 is a further perspective cut-away view of FIG. 8;

FIG. 13 is a front view of a housing element and cover engagement and placement assembly of a medical instrument cover dispensing apparatus wherein a cover receiving portion of the cover engagement and placement assembly is in a second configuration;

FIG. 14 is a sectional view of FIG. 13;

FIG. 15 is a perspective view of FIG. 13;

FIG. 16 is a perspective cut-away view of FIG. 13;

FIG. 17 is a sectional view of a feed arrangement, housing element, and cover engagement and placement assembly of a medical instrument cover dispensing apparatus wherein the feed arrangement is in a first configuration;

FIG. 18 is a detail view of detail A of FIG. 17;

FIG. 19 is a perspective cut-away view of FIG. 17;

FIG. 20 is a sectional view of a feed arrangement, housing element, and cover engagement and placement assembly of a medical instrument cover dispensing apparatus wherein the feed arrangement is in a second configuration;

FIG. 21 is a detail view of detail B of FIG. 20;

FIG. 22 is a perspective cut-away view of FIG. 20;

FIG. 23 is a front view of a cover urging element and associated biasing feature of a medical instrument cover dispensing apparatus;

FIG. 24 is a sectional view of FIG. 23;

FIG. 25 is a front view of a cover storage cartridge of a medical instrument cover dispensing apparatus;

FIG. 26 is a sectional view of FIG. 25;

FIG. 27 is a perspective view of a cover engagement and placement assembly of an embodiment of a medical instrument cover dispensing apparatus comprising a rib removal feature;

FIG. 28 is a front view of FIG. 27;

FIG. 29 is a sectional view of FIG. 27;

FIG. 30 is a front view of a housing element of an embodiment of a medical instrument cover dispensing apparatus showing throughbores in a guide formation thereof for receiving a biasing means of a de-seat feature thereof;

FIG. 31 is a perspective view of FIG. 30;

FIG. 32 is a front view of a feed arrangement of an embodiment of a medical instrument cover dispensing apparatus showing throughbores formed in spacing members for receiving a biasing element of a cover retaining and release feature thereof;

FIG. 33 is a perspective view of FIG. 32;

FIG. 34 is a perspective view of an alternative embodiment of a housing element and de-seat feature of a medical instrument cover dispensing apparatus;

FIG. 35 is a side view of FIG. 34;

FIG. 36 is a sectional view of section AA of FIG. 35;

FIG. 37 is a detail view of detail C of FIG. 36;

FIG. 38 is a perspective view of an alternative embodiment of a housing element and de-seat feature of a medical instrument cover dispensing apparatus showing a medical instrument cover located such that an outer rib thereof is locatable in a seat feature of said housing element;

FIG. 39 is a side view of FIG. 38;

FIG. 40 is a sectional view of section BB of FIG. 39;

FIG. 41 is a detail view of detail D of FIG. 40;

FIG. 42 is s side view of an embodiment of medical instrument cover for use in a medical instrument cover dispensing apparatus;

FIG. 43 is a sectional view of section CC of FIG. 42;

FIG. 44 is side view a plurality of the medical instrument covers of FIG. 42 in a stacked arrangement; and

FIG. 45 is a sectional view of section DD of FIG. 44.

### DETAILED DESCRIPTION OF THE DRAWINGS

The present teaching will now be described with reference to an exemplary medical instrument cover dispensing apparatus. It will be understood that the exemplary medical instrument cover dispensing apparatus is provided to assist in an understanding of the present teaching and are not to be construed as limiting in any fashion. Furthermore, elements or components that are described with reference to any one Figure may be interchanged with those of other Figures or other equivalent elements without departing from the spirit of the present teaching.

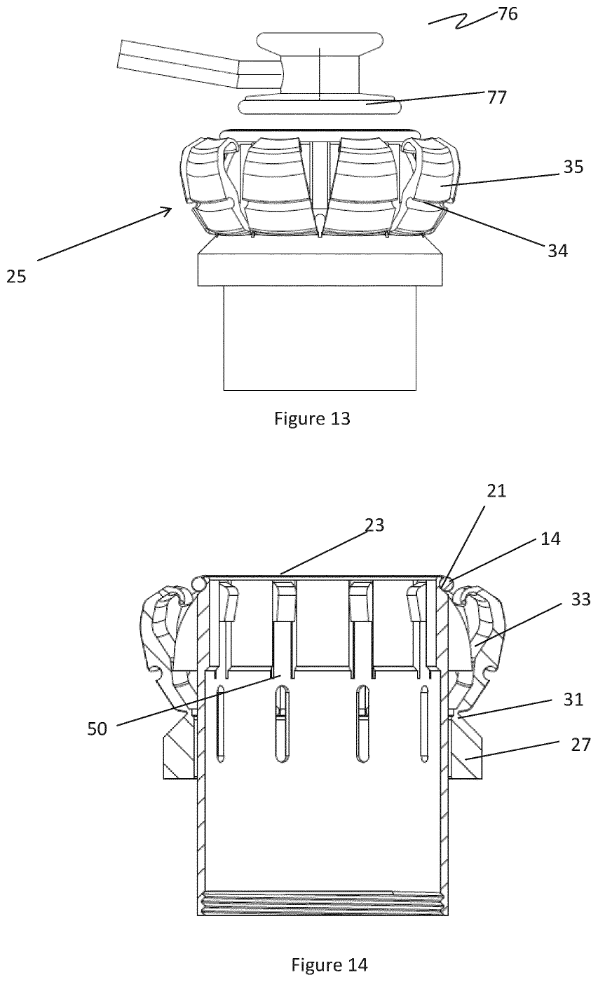

Referring now to the Figures there is illustrated a medical instrument cover dispensing apparatus 10 comprising a cover storage cartridge 11 configured to house a plurality of medical instrument covers 12 (in the description hereinafter referred to as a cover). The cover storage cartridge 11 may house any reasonable number of medical instrument covers 12, and in preferred embodiments houses over one hundred medical instrument covers 12. In the preferred embodiment, as shown in the drawings, the medical instrument is a stethoscope 76, and the medical instrument covers 12 are stethoscope covers 12. However, it should be understood that the invention could easily be adapted to dispense covers for various medical instruments. The covers 12 are in a first configuration when housed in the cover storage cartridge 11. The apparatus 10 further comprises a cover engagement and placement assembly 13 configured to receive a cover 12, or specifically an outer rib 14 thereof, from the cover storage cartridge 11, and move said cover 12 such that said cover 12 is placed in a second configuration wherein it is operably engagable by a stethoscope 76. A cover 12 in the second configuration can be seen in FIGS. 13 to 15. The apparatus 10 is configured to supply one cover 12 at a time such that a user can operably engage the cover 12 with a stethoscope 76 in a manner which acts to cover the stethoscope 76 without the user touching the cover 12. Advantageously, covers 12 may be applied to the stethoscope 76 without contact which could contaminate the cover 12. The cover storage cartridge 11 is a removable cartridge 11. Advantageously, the apparatus 10 may be replenished with stethoscope covers 12 by insertion of a single cartridge 11 containing a plurality of covers 12, thus preventing the need to reload loose covers or uncontained stacks of covers which further reduces the requirement to touch the covers 12 and the likelihood of droppage during loading. Thus, the likelihood of contamination of the covers 12 is reduced. Moreover, loading of loose covers 12 or uncontained stacks of covers 12 can be intricate and time consuming. The apparatus 10 further comprises a feed arrangement 15 which feeds covers 12 from the cover storage cartridge 11 to the cover engagement and placement assembly 13. The specifics of the feeding operation are further described below. The apparatus 10 further comprises a housing element 16 having an outer wall portion 17 defining an interior housing space 18, the housing element also having an upper opening 19 and a lower opening 20 opposing the upper opening 19. The upper opening 19 of the housing element 16 is an opening 19 which, when the apparatus 10 is assembled for use, is proximal the end of the apparatus 10 from which covers 12 are dispensed. The outer wall portion 17 of the housing element 16 comprises a seat feature 21 configured to receive the outer rib 14 of a cover 12. FIG. 14 shows the outer rib 14 of the cover 12 located in the seat feature 21. As can be seen, for example, in FIG. 31, the seat feature 21 is a circumferential recess 21 formed in the outer wall 17 of the housing element 16, proximal to the upper opening 19 thereof.

In use, the cover storage cartridge 11 and feed arrangement 15 are disposed within the interior housing space 18 of the housing element 16. The housing element 16 is a generally cylindrical element 16 and as such defines a generally cylindrical interior housing space 18. The feed arrangement 15 is also generally cylindrical and sized such that it is insertable into the cylindrical inner housing space 18 of the housing element 16. The feed arrangement 15 also defines a generally cylindrical inner space 22 which is sized such that the cover storage cartridge 11 is insertable thereinto. The housing element 16, feed arrangement 15, and cover storage cartridge 11 are disposed co-axially in relation to each other. In addition, the cover engagement and placement assembly 13 is disposed co-axially in relation to the housing element 16. Relative movement between any of the cover engagement and placement assembly 13, housing element 16, feed arrangement 15, and/or the cover storage cartridge 11 is generally movement along the common axis of these elements 11, 13, 15, 16. The common axis may be defined as extending in the axial direction of the generally cylindrical housing element 16. The cover storage cartridge 11 is also generally cylindrical in shape and defines a generally cylindrical cover receiving space 52 configured to receive the generally circular covers 12. However, it should be understood that the covers 12 may be of any reasonable shape such as but not limited to oval, a combination oval and circular shape, an irregular circular shape. It should also be understood that the general shape of the cover storage cartridge 11, feed arrangement 15, housing element 16, and/or cover engagement and placement assembly 13 could be easily adapted by the skilled person to accommodate differing shapes of covers 12. As such, the scope of the present invention extends to the apparatus as herein described which can dispense covers 12 of any shape, and also extends to covers 12 of any shape.

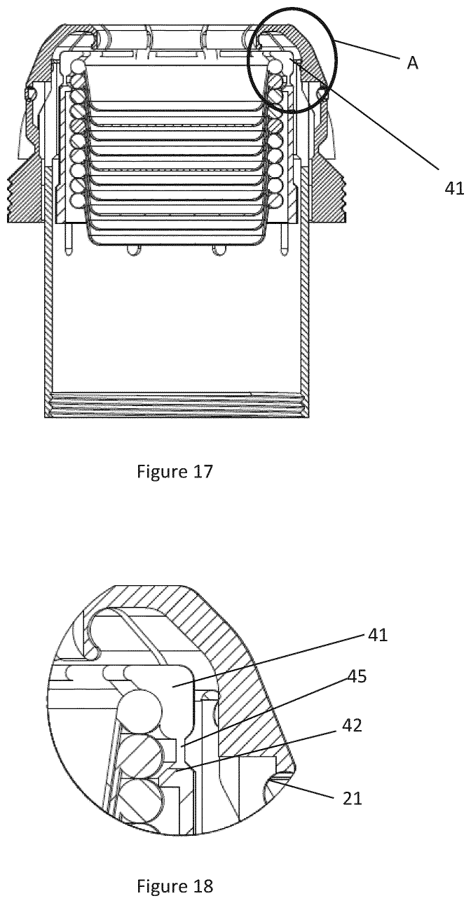

The cover storage cartridge 11 and feed arrangement 15 are movably mountable within the interior housing space 18 of the housing element 16. The cover engagement and placement assembly 13 is attachable to the feed arrangement 15 such that it is movable with the feed arrangement 15. The cover 12 is a generally circular cover 12 having a circular outer rib 14 defining a central aperture. A membrane 23 is formed integrally of said outer rib 14 and spans the central aperture. In the first configuration, as can be seen for example in FIG. 4, the Cover 12 forms a cup like shape with the outer rib 14 forming an upper lip 14 and the membrane 23 extending therebelow to form a cup. Advantageously, as can be seen for example in FIG. 17, the covers 12 can be stacked to conserve space in the cover storage cartridge 11. In the second configuration, as can for example be seen in FIG. 14, the cover 12 is stretched such that the membrane 23 is generally flat and in line with the outer rib 14.

As discussed above, the cover engagement and placement assembly 13 and the feed arrangement are operably engageable with each other. This operable engagement is made through a plurality of slotted openings 24 in the outer wall portion 17 of the housing element 16. As such, movement of the cover engagement and placement assembly 13 effects movement of the feed arrangement 15. Likewise, movement of the feed arrangement 15 effects movement of the cover engagement and placement assembly 13. The cover engagement and placement assembly 13 and feed arrangement may therefore move as one, on either side of the outer wall 17 of the housing element 16, relative to the housing element 16. In a preferred embodiment, the engagement between the cover engagement and placement assembly 13 and feed arrangement 15 comprises mechanical fixings which are fixed at a first end to the cover engagement and placement assembly 13, pass through the slotted openings 24 in the outer wall 17 of the housing element 16, and are fixable at a second end to the feed arrangement 15. These mechanical fixings may be screws, bolts, or the like, and are movable within the slotted openings 24.

The cover engagement and placement assembly 13 comprises a cover receiving portion 25 which extends at least partially across the upper opening 19 of the housing element 16 in a first configuration of the cover receiving portion 25. The cover receiving portion 25 is configured to receive the outer rib 14 of a cover 12. In the preferred embodiment, the cover receiving portion 25 comprises a hooked formation 26 into which the outer rib 14 may be received and thereafter retained. The cover engagement and placement assembly 13 further comprises a base portion 27. The base portion 27 is the portion of the cover engagement and placement assembly 13 which is in engagement with the feed arrangement 15 via the mechanical fixings through the slotted openings 24 in the housing element 16. The slotted openings 24 are shaped and dimensioned to guide/restrict movement of the mechanical fixings, and thus the feed arrangement 15 and cover engagement and placement assembly 13 which are both connected thereto. Specifically, the mechanical fixings are guided within the slotted openings 24 such that the upward and downward travel of the feed arrangement 15 and cover engagement and placement assembly 13 relative to the housing element 16 is limited by the longitudinal length of the slotted openings 24. Moreover, the mechanical fixings are sized such that the width of the slotted openings 24 limits lateral movement of the mechanical fixings therein, and as such limits lateral and rotational movement of the feed arrangement 15 and cover engagement and placement assembly 13 relative to the housing element 16. This maintains the movement of the feed arrangement 15 and cover engagement and placement assembly 13 generally co-axial relative to the axial direction of the housing element 16.

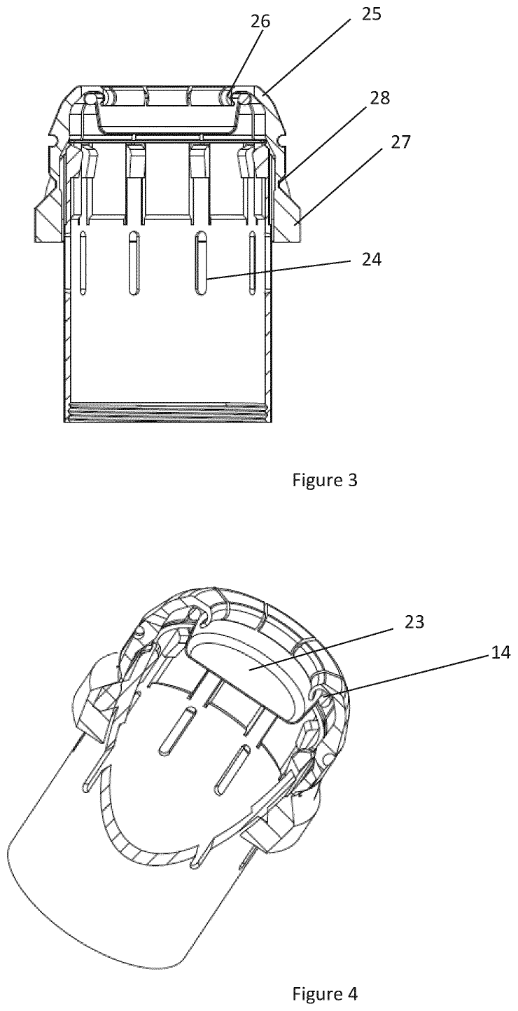

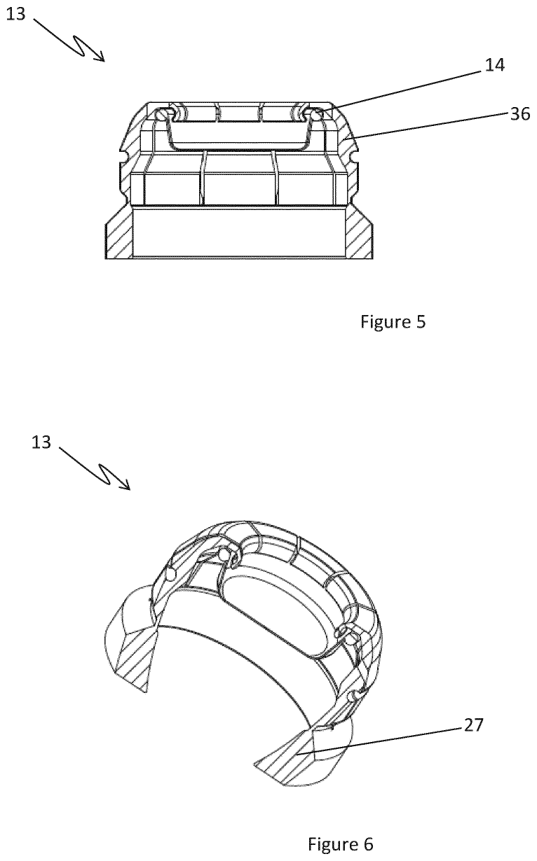

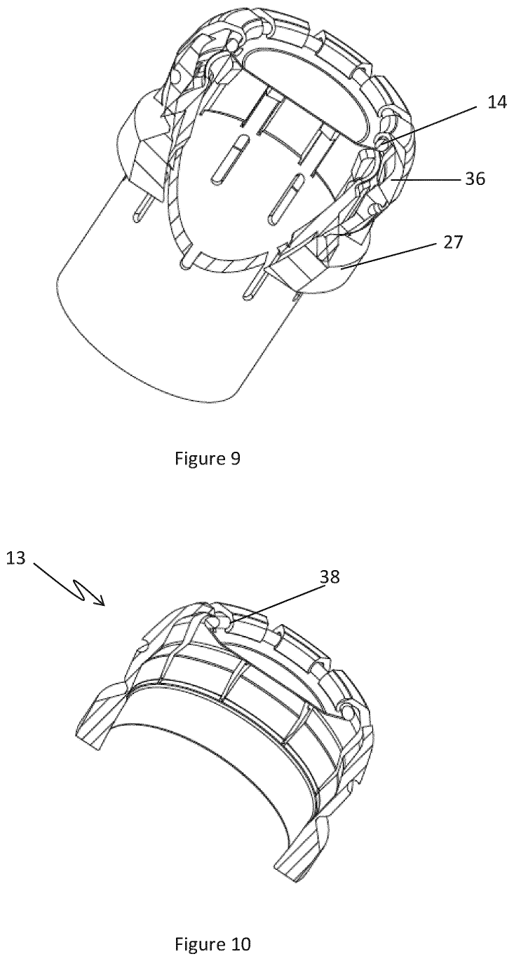

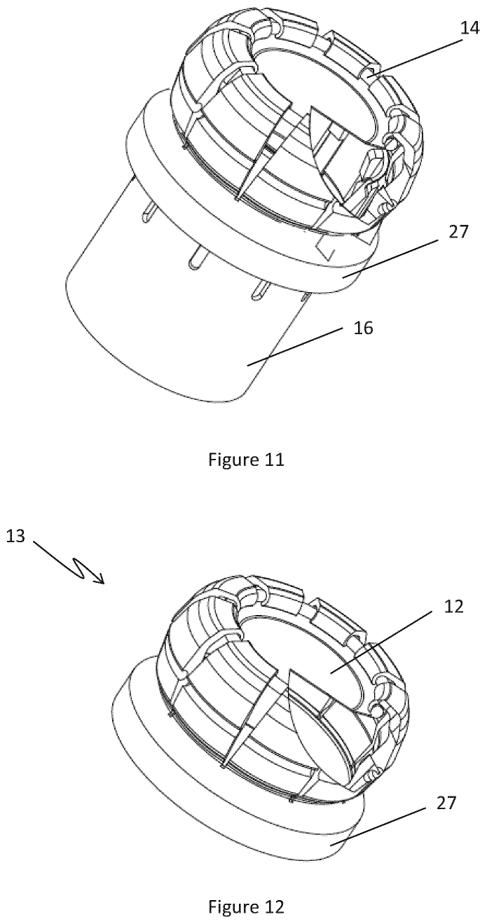

The cover receiving portion 25 of the cover engagement and placement assembly is in rotatable connection with the base portion 27 thereof via a hinged portion 28. The hinged portion 28 is preferably formed from the same material as the remainder of the cover engagement and placement assembly 13, said material being elastically deformable such that the hinged portion is optimised to bias the cover receiving portion 25 in its first configuration. More specifically, should the hinged portion 28 be deformed by a force, removal of said force will result in the hinged portion returning to its undeformed state. The undeformed state of the hinged portion 28 is the state which relates to the first configuration of the cover receiving portion 25. As such, when the cover receiving portion 25 is in its second configuration, or in a position between its first and second configurations, the elastically deformable nature of the hinged portion 28 acts to urge the cover receiving portion 25 back to its first configuration. As can be seen from the drawings, and in particular in FIGS. 3-16, the cover receiving portion 25 comprises a plurality of rotatable hook elements 36 arranged in a radial arrangement about the upper end 31 of the base portion 27. Each of the plurality of rotatable hook elements 36 has its own hinged portion 28 connecting it to the base portion 27 and the rotatable hook elements 36 move in unison between the first and second configurations as described in relation to the cover receiving portion 25. As can be seen from FIGS. 3-16, in the preferred embodiment, there are nine rotatable hook elements 36.

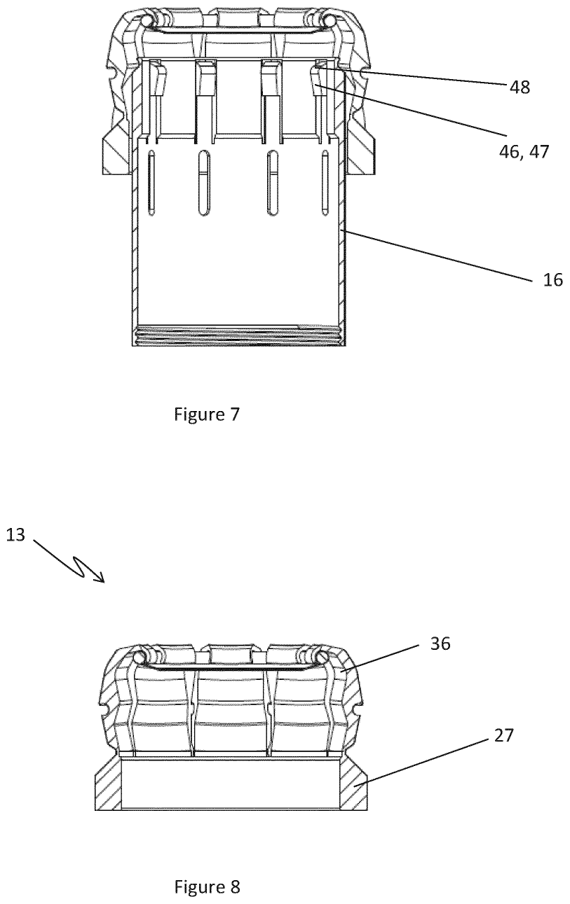

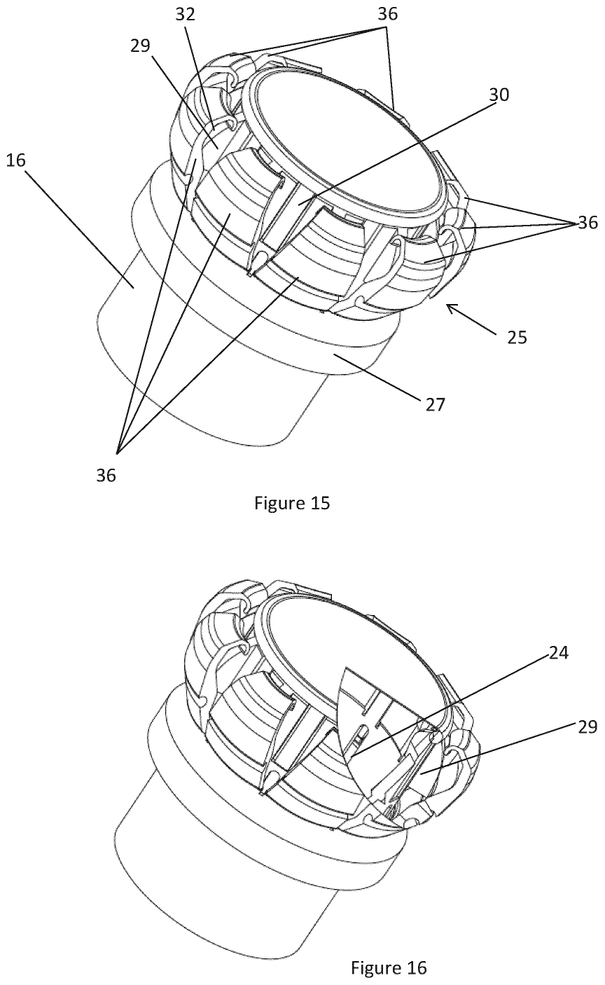

As can be viewed, for example, in FIG. 15, guide formations 29 are provided on the outer surface of the outer wall portion 17 of the housing element 16. The guide formations 29 are shaped and dimensioned to guide movement of the rotatable hook elements 36 from their first configuration wherein at least a portion thereof extends at least partially across the upper opening of the housing element 16 to their second configuration wherein the cover receiving portion 25 is rotated outwardly of the housing element 16. The guide formations 29 comprise arcuate guide formations which project from the outer surface 30 of the housing element 16 forming arcuate surfaces 32 which are engagable by an inner surface 33 of the rotatable hook elements 36 and along which the rotatable hook elements 36 are guided. Outward rotation of the rotatable hook elements 36 occurs as the engagement and placement arrangement 13 is moved downwards relative to the housing element 16. The movement of the engagement and placement arrangement 13 is best shown in FIGS. 3-16, wherein FIGS. 3-6 show the engagement and placement arrangement 13 in an upper position with the rotatable hook elements 36 thereof in their first configuration, FIGS. 7-12 show an initial downwards movement of the engagement and placement arrangement 13 and subsequent initial outwards rotation of the rotatable hook elements 36 thereof, and FIGS. 13-16 show the engagement and placement arrangement 13 in a lowermost position, wherein the rotatable hook elements 36 thereof are fully rotated outwardly such that said rotatable hook elements 36 are in their second configuration. In general, downwards movement herein refers to movement in the axial direction of the housing element 16 and in a direction towards the lower opening 20 thereof. The rotatable hook elements 36 may also comprise biasing elements (not shown) for urging the rotatable hook elements 36 inwards towards the outer surface 30 of the outer wall portion 17 of the housing element 16. In some embodiments, the biasing element is a spring or wire element (not shown) and the rotatable hook elements 36 comprise recesses 34 formed therein on an outer surface 35 thereof, the spring or wire element is receivable by said recess to at least partially span the plurality of rotatable hook elements 36. The spring or wire element is tensioned to urge or bias the rotatable hook elements 36 towards the outer surface 30 of the outer wall portion 17 of the housing element 16. The presence of the biasing element of the rotatable hook elements 36 may act to supplement the biasing/urging effect provided by the elasticity of the material of the hinged portion 28 connecting the base portion 27 of the cover engagement and placement assembly 13 to the rotatable hook elements 36 thereof, thus improving the functioning/reliability of the apparatus 10.

The outer rib 14 of the cover 12 is an elastically deformable outer rib 14. The rotatable hook elements 36 each comprise a receiving cup 38 sized and dimensioned to receive the outer rib 14 of the medical instrument cover 12. The cover engagement and placement assembly 13 is movable such that the rotatable hook elements 36 thereof pull the outer rib of an uppermost medical instrument cover 37 of the cover storage arrangement from a position generally above the interior housing space 18 of the housing element 16 and deposit said outer rib 14 into the seat feature 21 formed on the outer wall portion 17 of the housing element 16. The seat feature 21 is sized and dimensioned to receive the outer rib 14 of the cover 12. The membrane 23 of the cover 12 covers the upper opening 19 in the housing element 16 when the outer rib 14 of said cover 12 is locatable in the seat feature 21. The level of elasticity of the outer rib 14 of the cover 12 is toleranced such that said outer rib 14 becomes dislodged from the receiving cups 38 of the rotatable hook elements 36 and deposited into the seat feature 21 as the movement of the cover engagement and placement assembly 13 causes the outer rib 14 of the cover 12 held in the receiving cups 38 to align with the location of the seat feature 21. Alternatively, or in conjunction, a rib removal feature 39 is provided on the cover engagement and placement assembly 13 which is adapted to disturb the outer rib 14 such that it is actively dislodged from the receiving cups 38 and deposited into the seat feature 21. The rib removal feature 39 comprises movable fingers 39 which span the receiving cups 38 of the rotatable hook elements 36. The plurality of hook elements 36 are configured to move in unison between the first and second configurations of the cover receiving portion 25 and form a jaw like arrangement 25 wherein the jaw like arrangement 25 is closed in the first configuration and open in the second configuration.

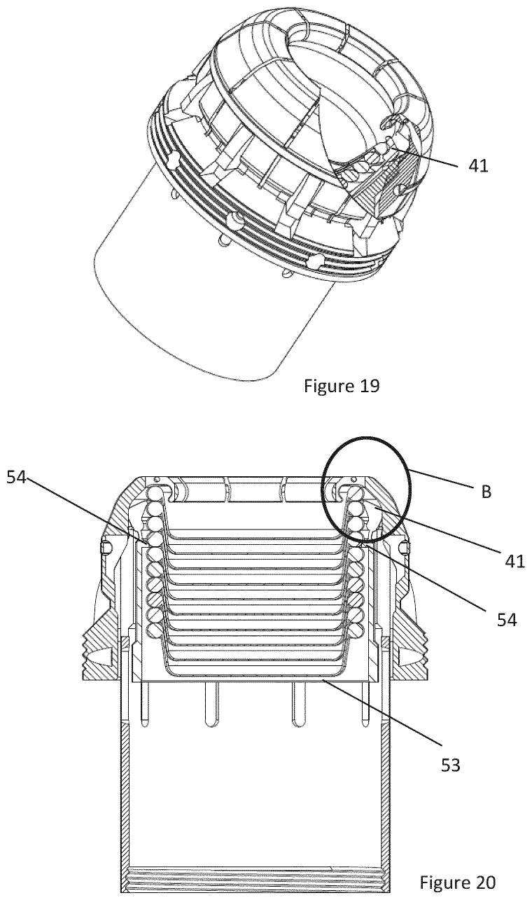

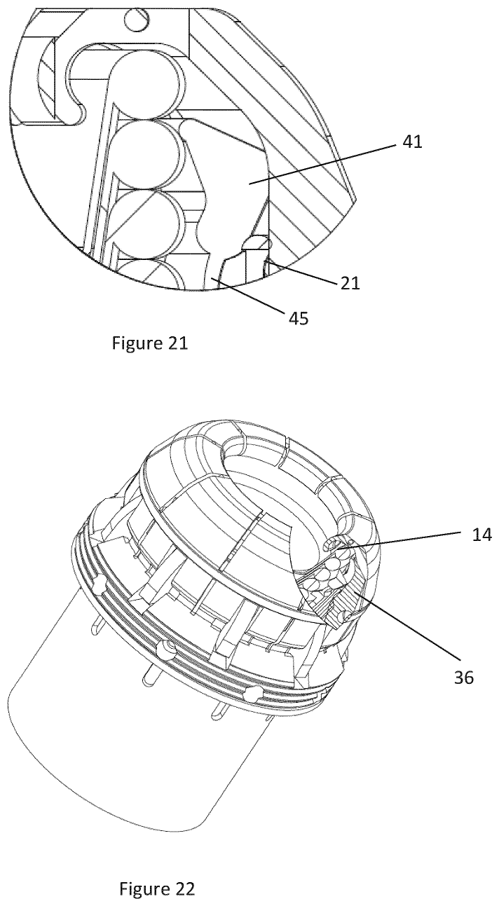

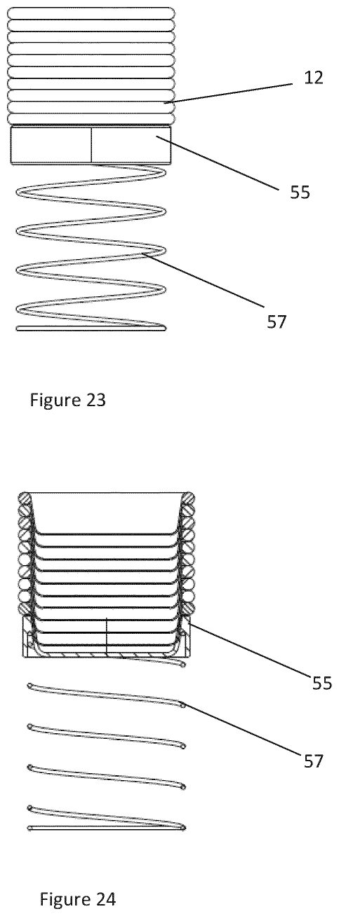

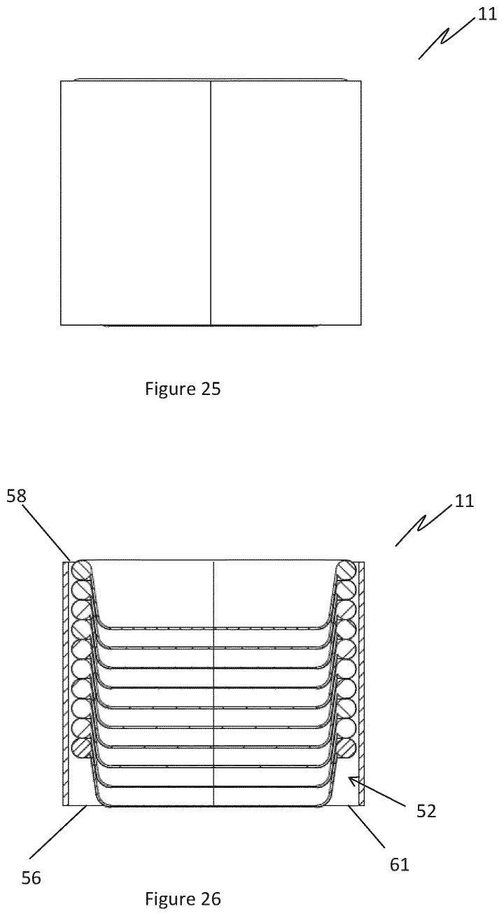

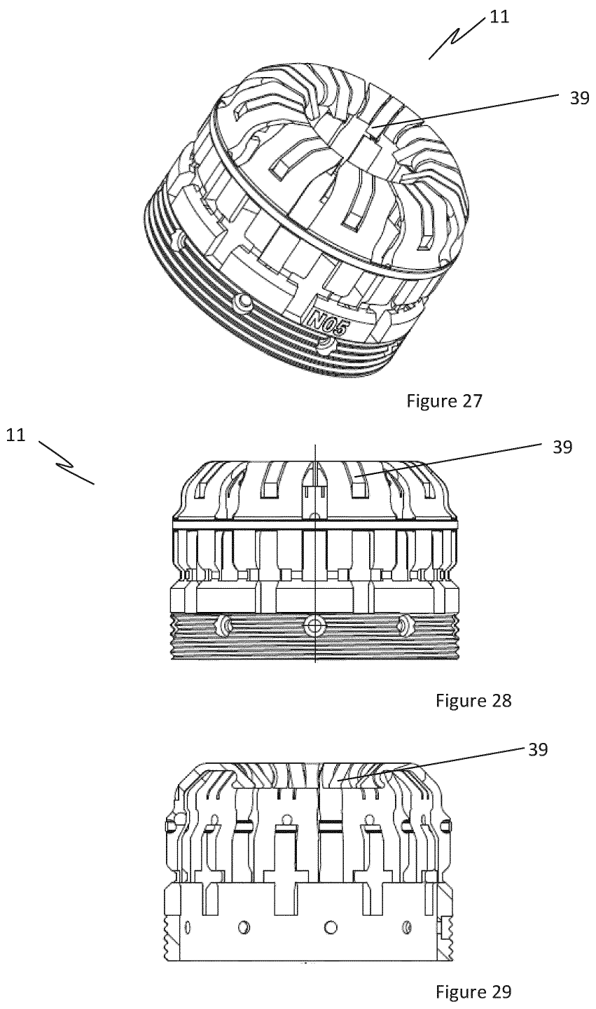

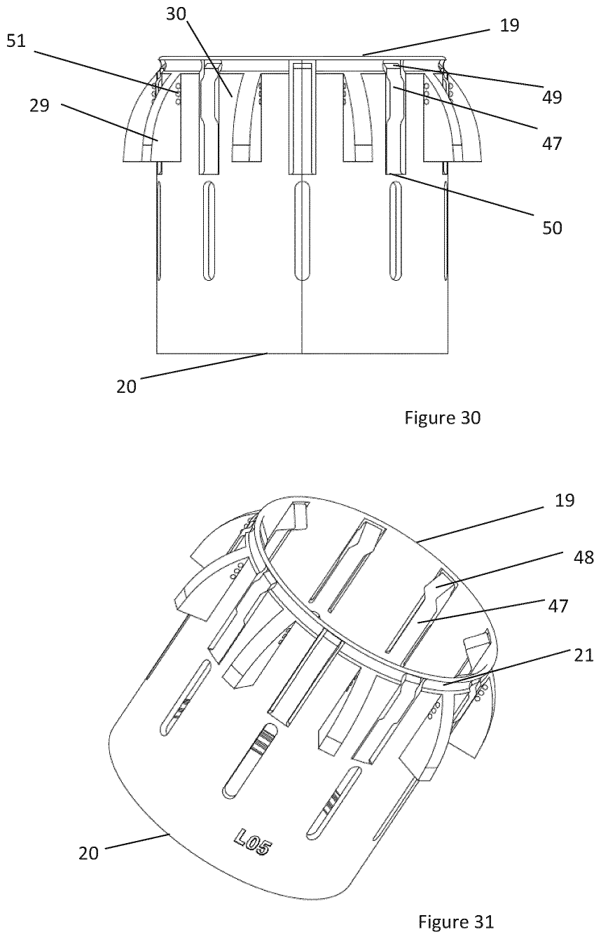

The feed arrangement 15 comprises an outer wall portion 40 defining its inner cylindrical space 22. As can best be viewed in FIGS. 17 to 22, and FIGS. 32 to 33, the outer wall portion 40 of the feed arrangement 15 further comprises one or more cover retaining and release features 41 at an upper end 42 thereof. The cover retaining and release features 41 are configured to selectably restrict upwards movement of the one or more covers 12 in the cover storage cartridge 11 when the feed arrangement 15 is in a first configuration. The first configuration of the feed arrangement 15 can be seen in FIGS. 17 to 19. When the feed arrangement 15 is in a second configuration, as can be seen in FIGS. 20-22, the one or more cover retaining and release features 41 release an uppermost cover 12 of the one or more covers 12 in the cover storage cartridge such that said uppermost cover 12 is permitted to travel towards and into contact with the hook elements 36 of the cover engagement and placement assembly 13. In the preferable embodiment, the feed arrangement 15 comprises a plurality of cover retaining and release features 41 disposed radially on the upper edge 42 thereof. As can be seen in FIG. 33, in the most preferable embodiment, there are nine cover retaining and release features 41. The plurality cover retaining and release features 41 are configured to move in unison between the first and second configurations of the feed arrangement 15. The plurality of cover retaining and release features 41 are disposed in a spaced apart radial arrangement on the upper edge 42 of the feed arrangement 15 and spacing members 43 are locatable therebetween. The cover retaining and release features 41 are rotatable between the first and second configurations thereof.

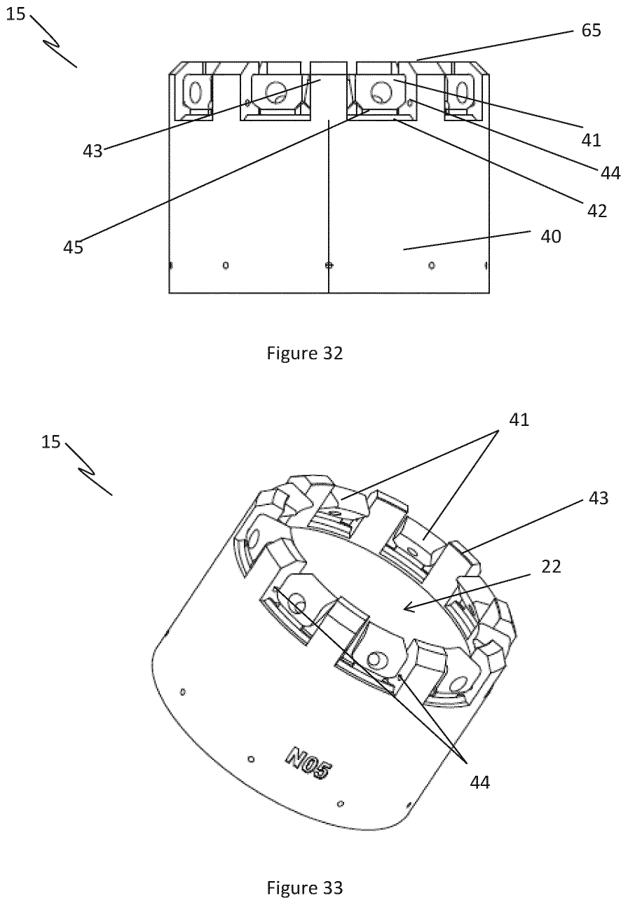

Movement of the feed arrangement 15 downwards relative to the housing element 16 causes the outer wall 17 of the housing element 16 to engage the cover retaining and release features 41 and cause rotation thereof inwardly towards the inner housing space 18 of the housing element 16, and into the first configuration from the second configuration. Movement of the feed arrangement 15 upwards relative to the housing element 16 causes movement of the cover retaining and release features 41 to move above, or move further above, the outer wall 17 of the housing element 16 such that the cover retaining and release features 41 may rotate outwardly relative to the inner housing space 18 of the housing element 16 and into the second configuration. The cover retaining and release features 41 may comprise a biasing element for urging/biasing the cover retaining and release features outwards relative to the inner housing space 18 of the housing element 16 and into the second configuration. The biasing element of the cover retaining and release features 41 comprises spring or wire element. As is best viewed in FIGS. 32 and 33, the spacing members 42 comprise throughbores 44 therein through which the spring or wire element of the biasing element of the cover retaining and release features 41 is threaded. The spring or wire element extends between the spacing members 42 on the side of the cover retaining and release features 41 proximal the upper opening 19 in the housing element 16. The spring or wire element of the biasing element of the cover retaining and release features 41 acts to exert a biasing force on the cover retaining and release features 41 outwards relative to the inner housing space 18 of the housing element 16. The cover retaining and release features 41 are in rotatable connection with the wall portion 40 of the feed arrangement thereof via a hinged portions 45. The hinged portions 45 are preferably formed from the same material as the remainder of the feed arrangement 15, however in any case, the material of the hinged portions 45 is elastically deformable such that the hinged portions 45 are optimised to bias the cover retaining and release features 41 outwardly in relation to the inner housing space 18 of the housing element 16. More specifically, should the hinged portion 45 be deformed by a force, removal of said force will result in the hinged portion 45 returning to its undeformed state. The undeformed state of the hinged portion 45 of the cover retaining and release features 41 is the state which relates to the second configuration of the cover retaining and release features 41. The effect of the hinged portions 45 may be supplemented by the biasing element of the cover retaining and release features 41 as described above to improve the functionality and reliability of the apparatus 10.

The housing element 16 comprises a cover de-seat feature 46 configured to urge an outer rib 14 of a cover 12 locatable in the seat feature 21 of the housing element 16 from said seat feature 21 and into engagement with a stethoscope 76. The cover de-seat feature 46 comprises a plurality of rotatable instrument engagement features 47 disposed radially around the outer wall 17 of the housing element 16 and projecting at least partially into the inner space 18 of the housing element 16 in a first configuration. The instrument engagement features 47 are rotatable into a second configuration wherein a portion 49 of the instrument engagement features 47 which are aligned with the seat feature 21 is rotated outwardly into the seat feature 21 such that the outer rib 14 of a cover 12 seated in the seat 21 feature is urged therefrom. Movement of the instrument engagement features 47 from the first configuration to the second configuration is effected by the insertion of a stethoscope 76 into the upper opening 19 of the housing element 16. A stethoscope 76 inserted into the upper opening 19 of the housing element 16 contacts the portion 48 of the cover de-seat feature which projects at least partially into the inner space of the housing element 16 in the first configuration, further insertion forcing the cover de-seat feature 47 to rotate outwardly relative to the inner space 18 of the housing element 16 and at least partially into the seat feature 21 such that an outer rib 14 of a cover 12 seated in the seat feature 21 is urged therefrom. The cover de-seat feature 47 is preferably formed from an elastically deformable material and connected to the outer wall 17 of the housing element 16 at a first end 50. Most preferably, the cover de-seat feature 47 is formed integrally of the housing element 16. The elastically deformable nature of the cover de-seat feature 47 results in rotation of the de-seat feature outwardly relative to the interior housing space 18 of the housing element 16 causing deformation of the material of the cover de-seat feature 47. Said deformation, being elastic in nature, results in a biasing or urging force which acts to urge/bias the cover de-seat feature back to its first configuration. Thus, when a stethoscope 76 is removed from the upper opening 19 of the housing element 16, the cover de-seat feature 47 is biased to return to its first configuration. The cover de-seat feature 47 may also comprise a biasing spring or wire element for biasing the cover de-seat feature 47 inwardly relative to the inner housing space 18 of the housing element 16 when the cover de-seat feature 47 is in the second configuration. In such embodiments, the guide formations 29 comprise apertures 51 therein and the de-seat spring or wire element is threadable therethrough such that the spring or wire element at least partially encircles the outer surface 30 of the outer wall of the housing element 16 at the location thereof having the de-seat features 47 such that said de-seat wire or spring element acts to exert a biasing force on the de-seat features 47 inwardly relative to the inner housing space 18 of the housing element. As stated, removal of a stethoscope 76 from the inner space of the housing element results in the cover de-seat feature 47 returning to the first configuration thereof.

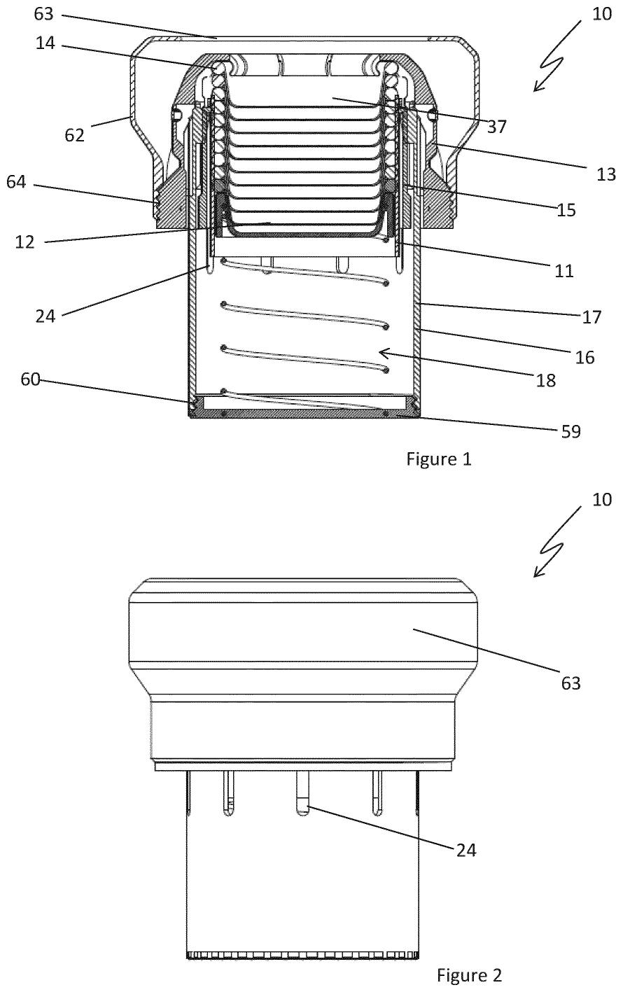

The cover storage cartridge 11 is sized and dimensioned for insertion into the feed arrangement 15. The cover storage cartridge 11 is insertable through a lower opening 53 in the feed arrangement 15 and the feed arrangement 15 comprises a stop feature 54 which engages with the cover storage cartridge 11 to stop the cover storage cartridge 11 at a desired location as it is inserted into the feed arrangement 15. The stop feature 54 is provided by a reduced interior diameter proximal a top end of the feed arrangement 15. A cover urging element 55 is provided, the cover urging element 55 being sized and dimensioned to be insertable into a lower opening 56 in the cartridge 11 and into contact with the covers 12 therein. The cover urging element 55 is also shaped to receive a cover 12 when the cover is in its first configuration, the cover urging element 55 being cup shaped. In this manner the cover urging element 55 may receive the lowermost cover in a stack of covers within a cover storage cartridge 11 and urge said lowermost cover, and therefore the remainder of the stack thereabove, upwards. The cover urging element 55 is in operable engagement with a spring 57 to bias the covers 12 towards an upper opening 58 of the cover storage cartridge 11. The spring is locatable between the cover urging element 55 and a base 59 of the housing element 16. The base 59 of the housing element 16 is a removable base 59 which is attachable to the housing element 16 to cover the lower opening 20 thereof via a mutually engagable threaded connection 60. However, it should be understood that this could be a snap on connection or any other such connection which would be well known to the skilled person. The spring 60 is compressed between the cover urging element and the base of the housing element. As can be best viewed in FIG. 1, the spring 57, at a first end, may be connected to the removable base 59 of the housing element such that removal of the removable base 59 also removes the spring 57 from the housing element 16. The cover urging element 55 is connected to the spring 57 at a second end of the spring distal the first end thereof. The cover storage cartridge 11 comprises a sacrificial retaining element 61 sized and dimensioned to cover the lower opening 56 of the cover storage cartridge 11. The sacrificial retaining element 61 prevents the covers 12 from falling from the lower opening 56 of the cartridge storage cover 11 before the cover storage cartridge 11 has been installed in the apparatus 10. The sacrificial retaining element 61 is formed from a material which my be punctured, perforated, tom, or the like by the cover urging element 55 as the cover urging element 55 passes into the lower opening 56 of the cover storage cartridge 11. The cover storage may also comprise an upper closure element for closing the upper opening 58 thereof. The upper closure element may be a peelable film element or the like which acts to prevent the medical instrument covers 12 from leaving the cover storage cartridge 11 via the upper opening 58 prior to the cartridge 11 being installed for use in the apparatus 10. The upper closure further acts to keep the medical instrument covers 12 clean and hygienic. When installing the cover storage cartridge 11 in the apparatus 10, the peelable film element is removed before the cartridge 11 is installed.

The medical instrument cover dispensing apparatus 10 further comprises a user engagable casing element 62. The user engagable casing element 62 is in operable engagement with the cover engagement and positioning arrangement 13 such that a user may move the cover engagement and positioning arrangement 13 by moving the user engagable casing element 62. The user engagable casing element 62 comprises a lower opening sized and dimensioned to receive the cover engagement and positioning arrangement 13 and an upper opening 63 to provide access for a stethoscope 76. The user engagable casing element is attachable to the base portion 27 of the cover engagement and positioning arrangement 13 via a bayonet type connection or via mutually engagable threaded connection 64. However, it should be understood that this could be a snap on connection or any other such connection which would be well known to the skilled person.

In use, the removable base 59 of the housing element 16 is removed and a cover storage cartridge, having covers 12 therein, is inserted into the housing element and into the feed arrangement 15 which is located within the housing element 16. The cover storage cartridge 11 is pushed upwards until it contacts the stop feature 54 of the feed arrangement 15. The removable base 59 is then replaced. As the spring 57 which acts to bias the cover urging element 55 is attached to the removable base 59 at a first end of the spring 57, replacement of the removable base 59 also results in placement of the spring 57, and cover urging element 55 which is attached to the second end of the spring 57, into the housing 16 such that the cover urging element receives the lowermost cover of the stack of covers 12 in the cover storage cartridge 11 and the spring 57 is compressed between the cover urging element 55 and the removable base 59. The spring 57 then acts to urge the stack of covers 12 in the cover storage cartridge 11 such that the uppermost cover in the stack contacts the cover engagement and placement assembly 13 and is in position to be engaged thereby. At this point, the cover storage cartridge 11 is installed in the apparatus 10 and the apparatus is ready to dispense covers. A user would then pull downwards on the user engagable casing element 63, moving it downwards relative to the housing element 16, and thus moving the feed arrangement 15 and cover engagement and placement assembly 13 downwards relative to the housing element 16. As the cover engagement and placement assembly 13 moves downward, the plurality of rotatable hook elements 36 thereof are forced by the guide formation 29 to rotate outwardly relative to the housing element 16. This rotation and downwards motion causes the rotatable hook elements 36 to pull the outer rib 14 of the topmost cover from the stack of covers 12 of the cover storage cartridge, and radially stretch the outer rib 14, pulling it towards the seat feature 21 in the outer wall 17 of the housing element 16. As the outer rib 14 approaches the seat feature 21, either the tension in the stretched outer rib, and/or the rib removal feature 39, cause the outer rib 14 to be deposited into the seat feature 21 from the rotatable hook elements 36. The outer rib 14 is then stretched around the outer surface 30 of the housing, with its associated membrane spanning the upper opening in the membrane. Simultaneous to the movement of the cover engagement and placement element 13 to stretch the outer rib 14, the feed arrangement also moves downwards by virtue of its connection to the cover engagement and placement assembly 13. This downwards movement of the feed arrangement 15 causes the cover retaining and release features 41 thereof to be forced by the upper edge of the wall portion 17 of the housing element 16 to rotate inwardly such that the cover below the uppermost cover is retained by the cover retaining and release features 41 and resultantly can not move upwards. This ensures that as the hook elements 36 are moved from a position wherein they prevent movement of the covers upwards from the cover storage cartridge 11, the covers do not make such an upwards movement and escape the apparatus 10. As discussed, at this stage of use, a cover is now stretched across the top of the housing element 16. In addition, a user may, at this stage of use, release the downwards force applied to the user engagable casing element 63 such that the cover engagement and placement assembly 13 and feed arrangement 15 are free to move upwardly until the hook elements 36 contact the outer rib 14 of the medical instrument cover 12 which is located in the seat feature 21, which prevents further movement of the cover engagement and placement assembly 13 and feed arrangement 15 until a user has removed the medical instrument cover 12 having said outer rib 14 in the seat feature 21. A user may now push a stethoscope head 77 into the membrane of the stretched cover 12, stretching the membrane 23 thereof. As the stethoscope head 77 moves downwards into the housing element 16, it contacts the de-seat features 47 thereof, forcing the de-seat features 47 to rotate outwardly such that the upper portion 49 thereof moved into the seat feature 21 and urges the outer rib 14 therein from the seat feature 21. The outer rib 14 which is urged from the seat feature 21 contracts due to its elastic nature and wraps around a backside of the stethoscope head, which is at this stage being held in the housing element 16 by a user as described. The user continues to push downwards until the stethoscope head contacts the upper end 65 of the spacing members 42 of the feed arrangement 15. The user may then remove the stethoscope head 77 from the apparatus, the stethoscope head 77 now having a cover 12 applied thereto. Upon the user removing the stethoscope head 77, the de-seat features 47 are free to rotate inwardly and are urged to do so by virtue of their elastically deformable nature returning the de-seat features 47 to their undeformed state, and/or by the biasing wire or spring thereof as described herein. At this stage, the cover engagement and placement assembly 13 and feed arrangement 15 are free to move upwards until they are in their first configurations as the outer rib 14 of the cover 12 which was preventing further upwards movement of these components 13, 15 has now been removed from the seat feature 21. The rotatable hook elements 36 are urged to move upwards and rotate inwardly, back along the guide formation 29. The urging of the rotatable hook elements is provided by the elastically deformable nature of the hinged portion 28 returning the hinged portion 28 to its undeformed state, and/or provided by the spring or wire biasing element of the rotatable hook elements 36 as described above. The rotatable hook members therefore move such that the cover engagement and placement assembly 13 is back to the first configuration wherein it may receive an outer rib 14 of a cover from the feed arrangement 15. As the feed arrangement 15 is attached to the cover engagement and placement assembly 13 it too moves upwardly as the cover engagement and placement assembly 13 moves upwardly. As the feed arrangement 15 moves upwardly, the cover retaining and release features 41 thereof move upwardly such that a sufficient portion thereof is above the outer wall portion 17 of the housing element 16 and therefore the cover retaining and release features 41 are free to rotate outwardly in relation to the housing element 16. Outward rotation of the cover retaining and release features 41 permits the remaining covers 12 in the cover storage cartridge 11 to move upwards, under the influence of the spring 57, such that the uppermost cover of the remaining covers 12 engages the rotatable hook elements 36 of the cover engagement and placement assembly 13 such that the apparatus is in a state to begin the cover dispensing process again. Dispensing of covers 12 is repeated until depletion of the covers 12 in the cover storage cartridge 11, at which point the empty cover storage cartridge 11 is replaced with a full cover storage cartridge 11.

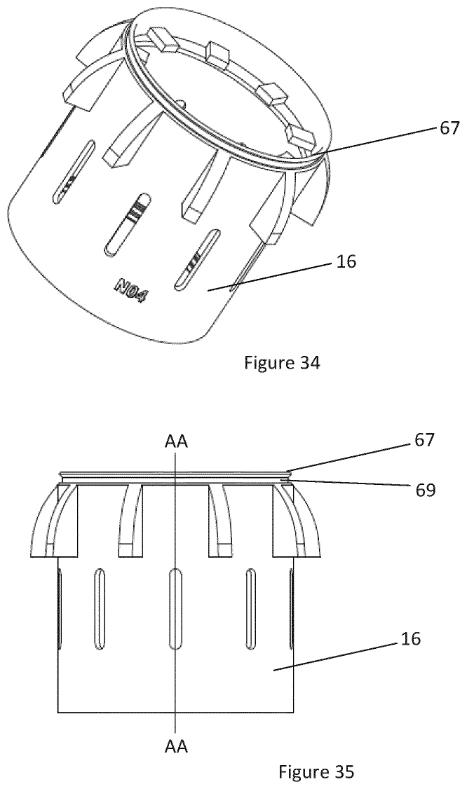

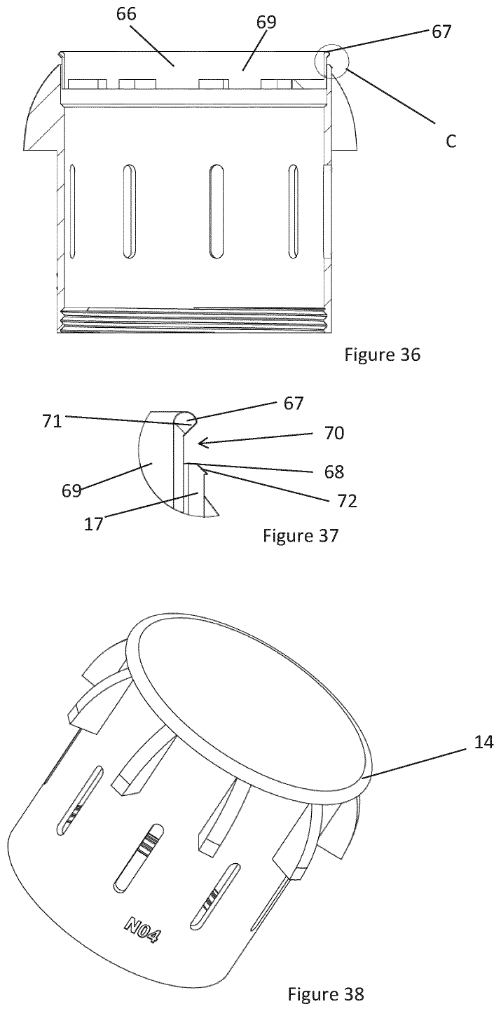

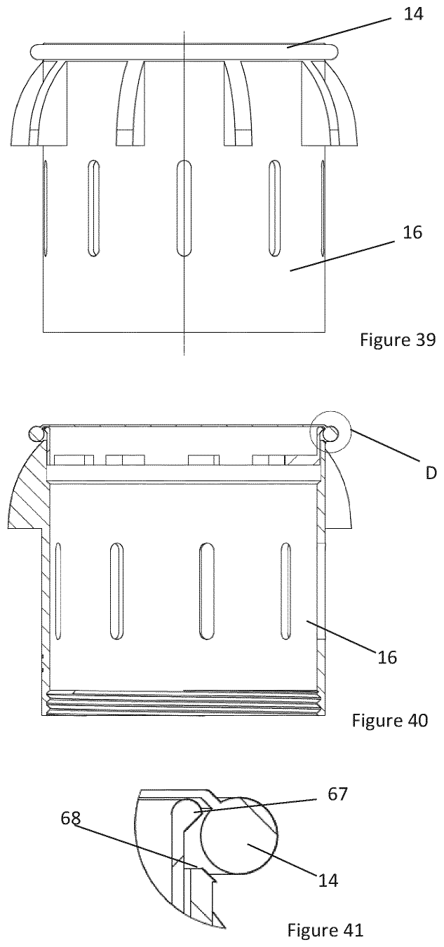

In an alternative embodiment, as can be seen in FIGS. 34 to 41, the cover de-seat feature comprises a slidable de-seat member 66. The slidable de-seat member 66 is slidably mountable relative to the housing element 16, and preferably is slidably mountable within the housing element 16. In the preferred case, the slidable de-seat member comprises a body portion 69. The body portion preferably being a circular element 69 sized and shaped such that it is at least partially insertable into the upper opening 19 in the housing element 16. The body portion 69 also has an aperture therein through which the inner housing space 18 of the housing element 16 can be accessed. The slidable de-seat member has a lip portion 67 which is selectably engagable with an upper end 68 of the outer wall portion of the housing element 16. The lip portion 67 is movable between first and second positions as the slidable de-seat member 66 slides upwards and downwards within the housing element 16. In the first position, the lip portion 67 is locatable such that the seat feature 70 is defined between the lip portion 67 and an upper end 68 of the outer wall portion 17 of the housing element 16. This can be best viewed in FIGS. 37 and 41. In the second position, the lip portion 67 is locatable such that it is in engagement with the upper end 68 of the outer wall portion 17 of the housing element 16. Alternatively, in the second position, the lip portion 67 does not actually engage with the upper end 68 of the outer wall portion 17 of the housing element 16, however the lip portion 67 is locatable such that the space between the lip portion 67 and said upper end 68 is reduced when compared to the space therebetween when the lip portion 67 is in the first position. Therefore, in use, and as can be seen in FIGS. 38 to 41, when the lip portion 67 is in a first position an outer rib 14 of a medical instrument cover 12 is receivable in the seat feature 70 defined between the lip portion 67 and an upper end 68 of the outer wall portion 17 of the housing element 16. Movement of the slidable de-seat member downwards within the housing element 16 such that the lip portion 67 thereof is in the second position causes the lip portion 67 to urge the outer rib from the seat feature 70 and into contact with a medical instrument. Preferably, the lip feature 67 is a circumferential flange 67 which extends around the outer surface of the body portion 69 at an upper edge thereof. The lip portion 67 and the upper end 68 of the outer wall portion 17 of the housing element 16 may comprise sloped surfaces or chamfers 71, 72 which act to form a generally cup shaped seat feature 70 when the lip portion 67 is in the first position, and may act to more easily guide/urge the outer rib 14 of a medical instrument cover 12 from said seat feature 70 when the lip portion 67 is moved towards the second position.

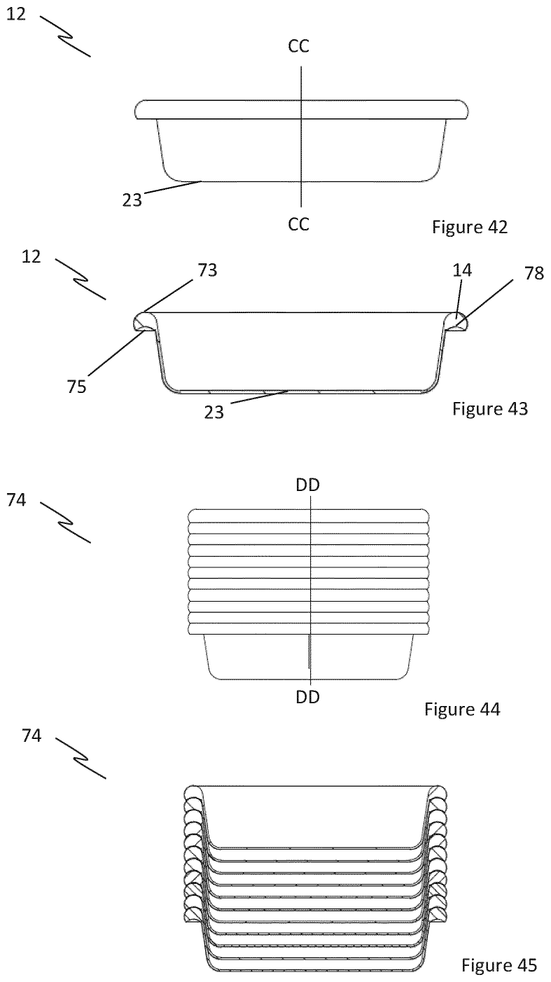

As can be seen in FIGS. 42 to 45, the medical instrument cover 12 may comprise a recess 78 formed on the underside 75 of the outer rib 14 thereof. The recess 78 in the outer rib 14 is shaped to receive the upper portion 73 of an outer rib 14 or another medical instrument cover 12 therein. The recess is a generally rounded or suitably shaped recess 78 formed for receiving the curved upper portion 73 of an adjacent medical instrument cover 12 when the covers are in a stacked arrangement 74. Advantageously, as can be seen in FIGS. 44 and 45, the medical instrument cover 12 may more easily be stacked with other medical instrument covers in a stacked arrangement 74.

Embodiments are also envisaged wherein the outer rib 14 of the medical instrument cover 12 is transferred directly from the cover engagement and placement assembly 13 to the stethoscope head 77 without first depositing said outer rib 14 in a seat feature.

Embodiments are also envisaged wherein the cover storage cartridge 11 may be installed by insertion through the upper opening 19 of housing element 16.

It should be understood that, the invention is described herein as implanted purely mechanically. However, the skilled person would understand that any of the movements accomplished by the apparatus 10 in dispensing covers 12 could be implemented electronically using suitable actuators, and may be controlled by electronics and/or software.

The invention is not limited to the embodiment(s) described herein but can be amended or modified without departing from the scope of the present invention.

1. A medical instrument cover dispensing apparatus comprising:
a cover storage arrangement configured to house one or more medical instrument covers, said one or more medical instrument covers being in a first configuration when housed in the cover storage arrangement; and
a cover engagement and placement assembly configured to receive one of the one or more medical instrument covers, or portion thereof, and move said received medical instrument cover such that said medical instrument cover is placed in a second configuration;
wherein, in the second configuration, said medical instrument cover is operably engagable by a medical instrument to attach the medical instrument cover to the medical instrument.

2. The medical instrument cover dispensing apparatus of claim 1 wherein the cover storage arrangement is a removable cover storage arrangement.

3. The medical instrument cover dispensing apparatus of claim 1 further comprising a feed arrangement for selectably feeding medical instrument covers from the cover storage arrangement to the cover engagement and placement assembly.

4. The medical instrument cover dispensing apparatus of claim 3 further comprising a housing element having an outer wall portion defining an interior housing space and an upper opening, the outer wall portion of the housing optionally comprising a seat feature and preferably, in use, the cover storage arrangement and feed arrangement being disposed at least partially within the interior housing space of the housing element; and optionally
wherein the cover engagement and placement assembly is movably mountable on the outer wall portion of the housing element, and further optionally
wherein the medical instrument cover is a generally circular cover having a circular outer rib defining a central aperture, and a membrane attached to or formed integrally with said outer rib and spanning the central aperture, and further optionally
wherein the housing element, feed arrangement, and cover engagement and placement assembly are generally cylindrical elements configured to be operable with the generally circular medical instrument cover.

5. (canceled)

6. (canceled)

7. (canceled)

8. The medical instrument cover dispensing apparatus of claim 4, wherein the cover engagement and placement assembly and the feed arrangement are operably engageable with each other such that movement of one of the cover engagement and placement assembly or feed arrangement causes movement of the respective other arrangement.

9. The medical instrument cover dispensing apparatus of claim 5, wherein the cover engagement and placement assembly comprises a cover receiving portion which extends at least partially across the upper opening of the housing element in a first configuration, the cover receiving portion being configured to receive an outer rib of a medical instrument cover, the cover engagement and placement assembly further comprising a base portion, wherein the cover receiving portion is in rotatable connection with the base portion, and optionally
wherein a guide formation is provided on the outer surface of the outer wall portion of the housing element the guide formation being shaped and dimensioned to guide movement ofthe cover receiving portion from a first configuration wherein at least a portion thereof extends at least partially across the upper opening of the housing element to a second configuration wherein the cover receiving portion is rotated outwardly of the housing element; and further optionally
wherein the engagement and lacement arrangement comprises urging means for urging the cover receiving, portion inwards towards the outer surface of the outer wall portion of the housing element, optionally the urging means being a biasing element.

10. (canceled)

11. (canceled)

12. The medical instrument cover dispensing apparatus of claim 6, wherein the cover engagement and placement assembly is movable such that the cover receiving portion thereof is configured to pull the outer rib of an uppermost medical instrument cover from a position generally above the interior housing space of the housing element and deposit said outer rib into the seat feature formed on the outer wall portion of the housing element.

13. The medical instrument cover dispensing apparatus of claim 6, wherein the cover receiving portion comprises a plurality of rotatable hook elements disposed radially on the base portion, the plurality of rotatable hook elements being configured to move in unison between the first and second configurations of the cover receiving portion.

14. The medical instrument cover dispensing apparatus of claim 3, wherein the feed arrangement comprises an outer wall portion defining an inner through channel sized and dimensioned for receiving the cover storage element, the outer wall portions further comprising one or more cover retaining and release features at an upper end thereof configured to selectably restrict upwards movement of the one or more medical instrument covers in the cover storage arrangement.

15. The medical instrument cover dispensing apparatus of claim 9, wherein the at least one cover retaining and release feature is rotatable between first and second configurations and, optionally, wherein movement of the feed arrangement downwards relative to the housing element causes the outer wall of the housing element to engage the at least one cover retaining and release feature and cause rotation thereof inwardly towards the inner housing space of the housing element and into the first configuration from the second configuration.

16. The medical instrument cover dispensing apparatus of claim 9 wherein movement of the feed arrangement upwards relative to the housing element effects movement of the at least one cover retaining and release feature to at least partially move out of the upper opening of the housing element and above the outer wall of the housing element such that the at least one cover retaining and release feature may rotate outwardly relative to the inner housing space of the housing element and into the second configuration.

17. The medical instrument cover dispensing apparatus of claim 11 wherein the at least one cover retaining and release feature comprises urging means for urging the cover retaining and release feature outwards relative to the inner housing space of the housing element and into the second configuration, optionally the urging means being a biasing element.

18. The medical instrument cover dispensing apparatus of claim 9, wherein the feed arrangement comprises a plurality of cover retaining and release features disposed radially on an upper end thereof.

19. The medical instrument cover dispensing apparatus of claim 3, wherein the housing element comprises a cover de-seat feature configured to urge the portion of a cover locatable in the seat feature of the housing element from said seat feature and into engagement with a medical instrument.

20. The medical instrument cover dispensing apparatus of claim 14, wherein the cover de-seat feature comprises a plurality of rotatable instrument engagement features disposed radially around the outer wall of the housing element and projecting at least partially into the inner space of the housing element in a first configuration, the cover de-seat feature being rotatable into a second configuration wherein a portion of the cover de-seat feature aligned with the seat feature is rotated outwardly into the seat feature such that a cover seated in the seat feature is urged therefrom.

21. The medical instrument cover dispensing apparatus of claim 14, wherein the cover de-seat feature comprises a slidable de-seat member, the slidable de-seat member being slidable relative to the housing element and sized and dimensioned to urge a cover seated in the seat feature therefrom.

22. The medical instrument cover dispensing apparatus of claim 3, wherein the cover storage apparatus is a cover storage cartridge, the cover storage cartridge comprising a cylindrical outer wall defining a cylindrical inner cover receiving space, the cover storage cartridge being sized and dimensioned for insertion into the feed arrangement; and optionally
wherein the cover storage cartridge is insertable through a lower opening in the feed arrangement, and the feed arrangement comprises a stop feature which engages with the cover storage cartridge to stop the cover storage cartridge at a desired location as it is inserted into the feed arrangement; and further optionally
wherein a cover urging element is provided, the cover urging element being sized and dimensioned to be insertable into a lower opening in the cover storage cartridge and into contact with medical instrument covers therein, the cover urging element being in operable engagement with a biasing feature to bias the medical instrument covers towards an upper opening of the cover storage cartridge and preferably, the biasing feature being a spring locatable between the cover urging element and a base of the housing element.

23. (canceled)

24. (canceled)

25. A cover storage arrangement for use in a medical instrument cover dispensing apparatus wherein the cover storage apparatus is a cover storage cartridge, the cover storage cartridge comprising a cylindrical outer wall defining a cylindrical inner cover receiving space sized and dimensioned to receive a plurality of medical instrument covers, the cover storage cartridge being sized and dimensioned for insertion into a feed arrangement of the medical instrument cover dispensing apparatus.

26. The cover storage arrangement of claim 18, wherein a lower opening of the cover storage cartridge is sized and dimensioned to receive a cover urging element such that the cover urging element may contact medical instrument covers therein, the cover urging element being in operable engagement with a biasing feature to bias the medical instrument covers towards an upper opening of the cover storage cartridge.

27. A medical instrument cover for use in a medical instrument cover dispensing apparatus, wherein the medical instrument cover comprises an outer rib defining a central aperture, and a membrane attached to or formed integrally of said outer rib and spanning the central aperture.
